(19) United States
(12) Patent Application Publication
(10) Pub. No.: US 2009/0082447 A1
(43) Pub. Date: Mar. 26, 2009

(54) Pharmaceutical Compositions Containing Omega 3 Fatty Acids

(75) Inventors: Didier Berthoumieu, Villenouvelle (FR); Jean-Francois Cordoliani, Sainte Foy D'Aigrefeuille (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: PIERRE FABRE MEDICAMENT, BOULOGNE (FR)

(21) Appl. No.: 12/086,678

(22) PCT Filed: Dec. 13, 2006
(86) PCT No.: PCT/FR2006/002728

§ 371 Date: Jun. 16, 2008

(30) Foreign Application Priority Data
Dec. 20, 2005 (FR) 0512917

(51) Int. Cl. A61K 31/202; A61P 25/28

(52) U.S. Cl. 514/560

(57) ABSTRACT

Pharmaceutical composition comprising a liquid oil rich in omega-3 polyunsaturated fatty acids which is absorbed on a silica-type support. The composition so obtained has the characteristics of a powder when the oil:silica ratio is of the order of 3:1 while at the same time allowing the required daily dose of omega-3 fatty acids to be delivered in one or more unit doses. The composition according to the invention can be used in the manufacture of oral pharmaceutical forms such as hard capsules.

The present invention relates to the field of oral pharmaceutical and nutraceutical specialties intended for the administration of oils rich in long-chain polyunsaturated fatty acids (LC-PUFAs) and more especially in omega-3 fatty acid.

LC-PUFAs are essential to the human diet. The LC-PUFA family includes omega-3 fatty acids and primarily docosahexaenoic acid (DHA, C22:6n-3) and eicosapentaenoic acid (EPA, C20:5n-3).

Such omega-3 fatty acids are essential components of cell membranes and as such are necessary for development and cerebral and cardiovascular function as well as for all aspects of tissue formation and repair. Many epidemiological studies have demonstrated the beneficial effect of omega-3 fatty acids on numerous pathological states, such as, for example, neurodegenerative diseases, arteriosclerosis, depression, blood lipaemia, skin diseases, hyperactivity in children or also rheumatisms.

Thus, in the field of Alzheimer's disease treatment, mnemonic capacity and mnesic function have been improved for periods of up to 6 months. Studies show that n-3 fatty acids are suitable for the prevention of, on the one hand, neurodegenerative diseases, especially vascular dementias, such as, for example, Alzheimer's disease, as well as, on the other hand and in a general manner, any disorders characterised by an impairment of cognitive functions (Analysis of dietary factors in Alzheimer's disease: clinical use of nutritional intervention for prevention and treatment of dementia. Otsuka M: Department of Neurology, Jichi Medical School, Omiya Medical Center. Nippon Ronen Igakkai Zasshi 2000 Dec. 37:970-3. Consumption of fish ans n-3 fatty acids and risk of incident Alzheimer disease. Morris & al. Arch. Neurol. 2003, 60, (7) :940-6. Fish, meat and risk of dementia: cohort study. BMJ 2002; 325: 932-933. Berberger-Gateau & al. Omega-3 fatty acids and risk of cognitive impairment and dementia. J. Alzheimers Dis. 2003, 5(4): 315-22).

In the field of depression, research shows that depressed people have reduced n-3 FA values. Since 1996, several studies have concluded that (n-3) fatty acids can have an anti-depressive effect and are to be recommended as a therapeutic supplement in schizophrenia (Arachidonic acid to eicosapentaenoic acid ratio in blood correlates positively with clinical symptoms of depression. Adams P B: Central Region Mental Health Service, Rockhampton Base Hospital, Queensland, Australia; Lawson S, Sanigorski A, Sinclair A J. Lipids 1996 Mar. 31 Suppl:S157-61).

There are also a number of studies demonstrating that the administration of DHA and EPA in amounts corresponding to the recommended intakes, especially in the form of fish oil, allows the level of triglycerides in the blood to be reduced and limits the formation of atheromatous plaque, all of which contributes to a reduction in the risk of fatal attack in populations having a cardiovascular risk.

It therefore appears that omega-3 fatty acids constitute an element essential to the good functioning of the cardiovascular and nervous systems and, since they cannot be synthesised, it is necessary for them to be provided by diet.

Those various studies have thus shown the value of supplementing the diet with a source of omega-3 fatty acid. Indeed the current diet, especially in western countries, is characterised by excessive consumption of saturated fatty acids to the detriment of LC-PUFAs, especially omega-3 fatty acids. The latter fatty acids are, in fact, synthesised by phytoplankton and are concentrated via the food chain in the flesh of oily fish, especially sardine, mackerel, anchovy, tuna or salmon. The way of life and dietary habits in the industrialised countries are the reasons why the consumption of foodstuffs rich in omega-3 fatty acids, especially oily fish, is low and at the very least insufficient to cover the recommended intakes of those fatty acids. It is thus clearly an established fact that it is necessary to supplement the diet to ensure optimum intake of omega-3 fatty acids. The recommended daily intake of omega-3 fatty acids corresponds to the equivalent of approximately from 1 to 5 grams of fish oil per day, this being dependent on the content of EPA and DHA in such an oil and also on the pathology targeted.

This awareness of the need to increase intakes of omega-3 fatty acids has led manufacturers to offer foodstuffs enriched with omega-3 as well as dietary supplements aimed at such supplementation.

In the pharmaceutical and nutraceutical field, the therapeutic virtues of omega-3 fatty acids in the various areas mentioned above have led to the development of compositions specifically intended for the treatment of cardiovascular pathologies via the oral administration of EPA and/or DHA in the form of fish oil that is enriched to a greater or lesser extent, or even of products that are more complex such as, for example, ethyl esters of those fatty acids.

Supplementing the diet with omega-3 fatty acid to counter the deficit in the traditional diet has been under development for some years. The supplementation is mainly effected by the oral administration of fish oil that is enriched to a greater or lesser extent with EPA and/or DHA.

The galenic forms intended for the oral administration of omega-3 fatty acids that are available on the market are in the form of hard or soft capsules filled with fish oil or fish oil derivative that is rich to a greater or lesser extent in omega-3. Generally, the oil may be in the form of triglycerides, fatty acid esters or a mixture of mono- or di-glycerides containing varying relative amounts of EPA and DHA.

Such hard or soft capsules represent the conventional and traditional form of formulation for fish oils, or derivatives, for oral administration. Indeed such forms allow the physicochemical properties of the oils present to be well preserved while at the same time ensuring a certain ease of use by the patient.

The casing of such hard or soft capsules generally consists of bioabsorbable polymers, such as gelatin or cellulose derivatives that rapidly disintegrate in aqueous media. Once ingested, therefore, the said capsules release the oil in the stomach following disintegration of the casing. The oil thereby released is subsequently absorbed in an analogous manner to any other fatty substance in the intestine. The omega-3 fatty acids thus rapidly end up in the blood circulation in the form of circulating triglycerides.

Such hard or soft capsules containing fish oil or fish oil derivative do not only have advantages however.

In fact, as indicated hereinbefore, once in the stomach the content of such a capsule is rapidly discharged and is absorbed along with the food intake containing other dietary lipids. That lipid discharge may cause a blood triglyceride peak shortly after absorption. Such a large non-delayed release of lipids may also cause digestive problems, especially in people whose bile function is not optimal.

In view of their sensitivity to oxidation due to the presence of a plurality of unsaturations, these fatty acids are especially sensitive to oxidation, which may bring about the formation of peroxidation products and volatile products having a particularly unpleasant odour. Irrespective of the presence of peroxidation products, fish oils generally do not have a very pleasant odour for the patient or consumer.

Also, in view of the release in the stomach when the hard or soft capsules rupture, gastro-oesophageal reflux is regularly noted in consumers of such formulations in the hours following absorption. Inevitably such reflux is a very negative experience not so much for the consumer himself as for those around him.

On an industrial scale, and more especially in terms of manufacturing processes, fish oils are not easy to handle. There are therefore drawbacks to the transport, treatment and handling of such oils both prior to and during the processes for manufacturing and filling the hard or soft capsules. Indeed, filling the soft or hard capsule with liquids, moreover oils, necessitates equipment and processes that are specific and generally expensive. In addition, in view of the tendency of omega-3 fatty acids towards oxidation, the handling and storage of fish oils or derivatives requires the use of anti-oxidants and/or measures and devices suitable for the protection of these products.

In order to overcome the problems of reflux and of release of the fish oils, technological solutions, such as, for example, encapsulation, have been proposed. Such technology remains expensive and complex however. In addition, in view of the ratio of oil to encapsulation material of the order of 1:4, the galenic forms obtained do not allow the required amount of oil to be delivered in a form and size suitable for the patient. Indeed, given that the average recommended daily intake of fish oil is of the order of from 1 to 3 grams, once that is encapsulated and mixed with the customary excipients daily doses for ingestion by the patient are of the order of 10 grams, which may represent a plurality of administrations, and that is not necessarily desirable.

The document WO2004/073689 describes the use of silica or a silica derivative for the formulation of a pharmaceutical composition by sorption of materials having an oily character containing a dissolved or dispersed active ingredient. The active ingredients in question are compounds of low water-solubility or of low bioavailability. In that document, the material of oily character is described as the vector fluid of the water-insoluble active ingredient. In addition it is stated that, in the context of medicament administration, the material of oily character must be therapeutically and chemically inert. To that end, the oily materials described are waxes, polyols, poloxamers, or even vegetable oils of the soybean, maize or peanut oil type, for example.

There are available on the market pharmaceutical and nutraceutical compositions comprising fish oils rich in omega-3 fatty acids that are absorbed on support materials, such as maltodextrins or cellulose derivatives. In addition to the fact that the absorption capacity of such materials is limited, however, the latter behave like a sponge and it becomes impossible to subject them to a compression step without observing the discharge of some or all of the absorbed oil.

It is therefore clearly apparent that there is a need for a galenic form specifically intended for the oral administration of a source of omega-3 fatty acid that does not have either the physiological or the technological drawbacks of hard or soft capsules filled with oil. One of the aims of the present invention is therefore to provide a galenic formulation that, on the one hand, provides for a progressive release and absorption of the omega-3 fatty acids and, on the other hand, limits the gastro-oesophageal reflux typically observed with the liquid oily forms. A further aim of the present invention is finally to provide a pharmaceutical composition containing a source of omega-3 fatty acids that is easy to handle on an industrial scale in manufacturing processes, for example. Finally, a further aim of the present invention is to provide a pharmaceutical or nutraceutical composition that allows the oral administration of an effective daily dose of omega-3 fatty acid in a manner that is convenient for the patient.

To that end, the present invention relates to a pharmaceutical composition containing a liquid oil rich in omega-3 fatty acid absorbed on a support comprising a silica or a silica derivative.

In the present description, and unless indicated otherwise, the fatty acid percentages are calculated and expressed as “surface area percentages” according to the European Pharmacopoeia method, monograph 1352.

In the context of the present invention, the expression liquid oil rich in omega-3 fatty acids relates to an oily fat in which the content of omega-3 fatty acids is of the order of at least 40%, preferably at least 55%, even at least 60% or even further at least 70%.

The omega-3 fatty acids of the composition according to the present invention may be in the form of a mixture of triglycerides, monoglycerides, diglycerides or other types of omega-3 fatty acid esters, such as ethyl esters, or even phospholipids such as, for example, phosphatidyl choline, phosphatidyl serine or phosphatidyl inositol.

Omega-3 fatty acids are understood to be fatty acids having an unsaturation in the (n-3) position. Preferably, it can be EPA or DHA, used alone or in admixture.

As a liquid oil rich in omega-3 fatty acids it will be possible to use a fish oil or fish oil mixture. Suitable fish oils are, for example, the oils of tuna, sardine, anchovy or salmon or of any other oily fish. Preferably, the fish oils rich in omega-3 contain approximately at least 40%, preferably at least approximately 55%, even at least 60% or even further at least 70% omega-3 fatty acid in the form of triglycerides.

By way of example, it will be possible to use a fish oil of the 18/12 type, that is to say having quantities of EPA of the order of 18% and of DHA of the order of 12%.

There also exist fish oils that are to a greater or lesser extent refined, concentrated, purified and/or enriched with omega-3 fatty acid, such as a 10/50 oil, that is to say an oil having relative amounts of EPA and DHA of the order of 10% and 50% respectively.

One of the main aspects of the present invention is the use of silica or a silica derivative as sorption material for an oil having a high content of omega-3 fatty acids. In the context of the present invention, the expression “silica or silica derivative” relates to the amorphous form of silicon dioxide in colloidal form or in the form of its derivatives, hydrated or non-hydrated, pyrogenic or non-pyrogenic, for example. Among the suitable silica derivatives there may be mentioned, for example, amorphous silica gels, hydrated colloidal silicas, pyrogenic hydrophilic silicas. Preference will be given to the use of an amorphous silica gel in hydrated colloidal form. Such compounds are traditionally used in the pharmaceutical, food, chemical or cosmetics industry for thickening liquids, reinforcing polymers and elastomers and facilitating the flow of powders, for example.

Those silicas or silica derivatives exhibit the property of absorbing liquids, this being the case in proportions ranging up to 200%, or even 300%, in percent by weight.

Use has thus been made of this property of those silicas, and accordingly another aspect of the present invention is the use of silica for the sorption of an oily source of omega-3 fatty acids as an active ingredient in the preparation of a pharmaceutical composition.

Silicas or silica derivatives, called “silica” hereinafter in the present description, are characterised generally by parameters such as the DOP oil uptake according to the standard NF30022.

In the context of the present invention, use will be made of silicas having a DOP oil uptake of between 100 and 300 inclusive, preferably of between 200 and 250 inclusive.

The silicas are in the form of small beads of greater or lesser porosity the size of which can vary from 1 to 6 μm, preferably from 2 to 5 μm, or even from 2 to 4 μm.

The BET surface area of the silicas that can be used in the context of the present invention may be between 300 and 600 m²/g inclusive, preferably between 350 and 500 m²/g inclusive, or even between 400 and 450 m²/g inclusive.

In the context of the present invention, any silica having a DOP oil uptake greater than about 150 will be suitable. Preference will be given to the use of colloidal silicon dioxides, especially a hydrophilic and/or hydrophobic colloidal silicon dioxide. A silica that is especially preferred is SYLOID® marketed by the company GRACE DAVISON.

In fact, the silica acts a little like a solid sponge. An aqueous or oily liquid is thus able to penetrate inside the three-dimensional structure of the silica beads and impregnate the said beads. In that way a composition is obtained that can be described as dry but of which the oil content can be of the order of from 150% to 200%, or even 300%, in percent by weight.

Accordingly, the ratio by weight of oil to silica may be between about 1.5:1 and about 3:1 inclusive, especially of the order of 2:1, this being the case to obtain a dry, more or less pulverulent form.

It will thus be possible for an oil rich in omega-3 fatty acid, such as a fish oil, to be absorbed by the silica, this being in an amount that may reach up to 300% in percent by weight.

It will accordingly be possible to obtain a composition according to the present invention delivering up to 19 mg of EPA and 95 mg of DHA in a dose of 330 mg, this being the case via the impregnation of silica to a level of 200% by weight with, for example, a traditional 10/50 fish oil. In the case of the present invention, it is the oily source material of omega-3 fatty acids, preferably a fish oil, that constitutes the active ingredient of the pharmaceutical composition.

The composition so obtained is in the form of a powder that is flowable and capable of being handled like any pharmaceutical powder.

Once absorbed within the three-dimensional structure of the silica particle, the oily source of omega-3 fatty acids will thus be enclosed, maintained and retained within the particle.

It will be possible by virtue of the rigid structure of the silica particles, which do not undergo deformation when a mechanical stress is applied to them, for the dry composition so obtained to be handled, poured and compressed in the presence of compression agents well known to the person skilled in the art without either seepage or discharge of oily material. It will thus be possible for the composition according to the present invention to be handled easily, and especially in equipment to be used for compression, this being in combination with compression adjuvants if needed. Examples of compression agents that may be mentioned are cellulose derivatives, lactose, polyols or calcium salts.

The impregnation of silica with the oily source may be effected in conventional manner and according to procedures known to the person skilled in the art.

The oily material and the silica may be mixed using a planetary-type or kneader-type mixer. The oil is progressively added to the silica in the course of mixing until the desired uptake of oil is reached.

In order to minimise the amount of air present within the silica particle, and hence oxidation, the impregnation can be carried out in vacuo or under an inert gas, for example. Alternatively, the impregnation can also be carried out using a supercritical fluid, such as CO₂, as vector fluidising the oily material. In addition, the use of supercritical CO₂ allows a homogeneous impregnation to be achieved while avoiding a gradient effect between the periphery and the heart of the silica particle.

The composition according to the present invention can thus be used in the preparation of unit-dose pharmaceutical forms intended for oral administration. Apart from the possibility of compressing the composition into the form of tablets which may, if desired, be film-coated to maximise protection against oxidation, the pulverulent composition according to the present invention can also be considered for use in the filling of soft or hard capsules.

The inventors of the present composition have noted that when the oil:silica ratio is increased beyond 3:1, the composition obtained becomes paste-like and is no longer pulverulent. Also, beyond that ratio of 3:1 and the more the quantity of oil increases, especially for ratios between 7:1 and 10:1 inclusive, the more the texture of the composition approaches a thixotropic gel. Hence, in the case of an oil:silica ratio of more than 3:1, and more especially a ratio between 5:1 and 10:1 inclusive, a composition according to the invention is obtained having a texture that ranges from a paste to a thixotropic gel. That rheological property, although different from the pulverulent form, in addition has numerous advantages both on the technological and the pharmaceutical level. In particular, the amount of oil delivered by a dose of such a composition will be greater. Furthermore, the gel form allows the delivery of a composition that can be used to fill soft capsules by pumping, for example.

The present invention relates to a pharmaceutical composition in which the liquid oil and the silica support on which that oil is absorbed represent approximately at least 80% by weight of said composition, preferably 90% and more preferably 95%. Indeed, it is that combination of oil and silica which constitutes the pharmaceutical composition as such, which is thus substantially composed of that combination of liquid oil absorbed on silica support. The liquid oil rich in omega-3 fatty acid represents the active ingredient of the present composition and the silica support is its functional support.

Thus, in a pharmaceutical composition according to the present invention, depending on the oil:silica ratio, the liquid oil represents approximately from 60 to 75% by weight, preferably from 65 to 70% by weight, of the total pharmaceutical composition according to the present invention, and the silica or the silica support represents approximately from 25 to 40% by weight, preferably 30 to 35% by weight, of the composition according to the present invention.

In the context of the present invention, the galenic forms so obtained, hard or soft capsules, typically contain a daily dose corresponding to the recommended intake of omega-3 fatty acid. Thus, by way of example, a hard capsule containing 200 mg of colloidal silica, impregnated with oil to a level of from 200 to 300% by weight, is able to deliver from 400 to 600 mg of fish oil, for example. Taking from 2 to 4 capsules daily will thus be sufficient to cover the recommended intake.

The capsules so obtained can be used for oral supplementation of omega-3 fatty acids. Thus, once taken and swallowed, those capsules release the composition in the stomach and it is possible to observe that the oil so impregnated is not suddenly discharged in the stomach or the intestine. In fact, it is the impregnated silica that is released. Accordingly, the effect observed is a progressive and prolonged release of the oil absorbed in that way.

In addition, since the oil has a tendency to be retained by the silica particles, which are taken along with the food bolus to the duodenum, gastro-oesophageal reflux, which is especially disagreeable for patients taking traditional oils in liquid form not absorbed on silica, is observed less.

Finally, the present invention relates also to the use, in the preparation of a medicament for use in the preventive and/curative treatment of neurodegenerative diseases, especially senile dementias, such as, for example, Alzheimer's disease, of a composition containing an oil rich in omega-3 fatty acids that is absorbed on silica.

### EXAMPLE 1

### 330 mg Hard Capsule of Silica Impregnated with 10/50 Fish Oil

An intimate mixture of 220 g of 10/50 fish oil with 110 g of SILOID® silica is prepared in a kneader-type mixer by the progressive addition of the oil to the silica. A silica impregnated to a level of 200% by weight is thereby obtained. Subsequently, 50 g of microcrystalline cellulose and 2.5 g of glycerol dibehenate are added and stirring is continued until a homogeneous pulverulent active mixture is obtained.

A compression-dosing device system is then used for filling the hard capsules. By way of compression-dosing devices, the active pulverulent mixture is taken from a hopper after the capsule has been opened (separation of the cap and the body). The compression-dosing device, through an alternative system, ejects the 382 mg dose of powder into the body of the capsule. The capsule is re-closed by fixing the cap on the body and is then clipped and finally ejected.

### EXAMPLE 2

### Direct-Compression Tablets

The production procedure for the pulverulent mixture is identical to that of the preceding Example, except that 250 g of microcrystalline cellulose are introduced instead of 50 g in order to facilitate compression.

Compression of the said pulverulent mixture obtained is carried out on a Korsch PH212 rotary press and 582.5 mg tablets having a diameter of 15 mm and a breaking strength of maximum 30N are obtained.

### EXAMPLE 3

### Oil Release by a Hard Capsule According to the Invention

A comparative disintegration test was carried out between a hard capsule according to Example 1 and a hard capsule containing the same oil but without silica. In both cases, the casing of the hard capsules is composed of pharmaceutical gelatin.

The capsules are introduced into the immersed analysis chamber of an ERWEKA ZT61D disintegration measurement apparatus immersed in water and thermostatically controlled at 37° C.

The content of the capsules is released after stirring for 5 minutes, subsequent to the dissolution of the casing material of the capsule.

It is to be noted that, in the case of the capsule filled with oil, the entire contents are to be found dispersed at the surface in the form of droplets.

Conversely, in the case of the capsule according to the present invention, only approximately 50% of the oil contained in the capsule is to be found floating at the surface, and that being in the form of far smaller droplets. The remainder of the oil is retained by the silica, some of which has been deposited at the bottom; another portion of the silica is in suspension in the water. When additional mixing is carried out, progressive release of the oil absorbed by the silica particles is observed.

1-7. (canceled)

8. A pharmaceutical composition comprising a liquid oil which is rich in omega-3 fatty acids, and absorbed on a support which comprises a silica or a silica derivative, wherein:
i. the ratio by weight of oil:silica or silica derivative is between 1.5:1 and 3:1 inclusive,
ii. the liquid oil constitutes approximately from 60 to 75% by weight of the pharmaceutical composition and
iii. the silica or the silica support constitutes approximately from 25 to 40% by weight of the composition.

9. The pharmaceutical composition of claim 8, wherein the silica or the silica derivative is selected from hydrated or non-hydrated colloidal silicas, pyrogenic or non-pyrogenic hydrophilic silicas and hydrated amorphous colloidal silica gels.

10. The pharmaceutical composition of claim 8, wherein the silica has a BET surface area of between 300 and 600 m²/g inclusive and a DOP oil uptake of between 100 and 300 inclusive.

11. The pharmaceutical composition of claim 8, wherein the loading of oil rich in omega-3 fatty acids in the support comprising the silica is between 150 and 300% by weight inclusive.

12. The pharmaceutical composition of claim 8, which is in an oral pharmaceutical form.

13. The pharmaceutical composition of claim 8, which is in a particulate, pulverulent or granular form.

14. A method for preventing or treating neurodegenerative diseases in a living animal body, including a human, comprising administering the pharmaceutical composition of claim 8 for the prevention or treatment thereof.

15. The method of claim 14, wherein the neurodegenerative disease is senile dementia.
